(19) United States
(12) Patent Application Publication
(10) Pub. No.: US 2007/0288001 A1
(43) Pub. Date: Dec. 13, 2007

(54) ENDOSCOPICALLY INTRODUCIBLE EXPANDABLE CAUTERY DEVICE

(76) Inventor: Pankaj Patel, Granger, IN (US)

Correspondence Address: BOTKIN & HALL, LLP, 105 E. JEFFERSON BLVD., SUITE 400, SOUTH BEND, IN, 46601, US

(21) Appl. No.: 11/761,127

(22) Filed: Jun. 11, 2007

Related U.S. Application Data
(63) Continuation-in-part of application No. 11/309,026, filed on Jun. 12, 2006.

(51) Int. Cl. A61B 18/12; A61B 1/00

(52) U.S. Cl. 606/49; 600/101

(57) ABSTRACT

An endoscopically introducible expandable cautery device having a probe slidably received in a tube. The probe has a cauterizing end terminating in a balloon which is expandable upon inflation with fluid supplied through a first lumen in communication with a source of fluid such as a syringe. The surface of the balloon has at least one electrical contact that provides a cautery effect when in contact with tissue to be treated. The balloon is inflatable to a size larger than the bore of an endoscope though which it may be introduced.

### CROSS REFERENCE TO RELATED APPLICATIONS

This application is a Continuation-in-Part and claims the benefit of U.S. Utility patent application Ser. No. 11/,309,026, filed Jun. 6, 2006, the disclosure of which is hereby incorporated by reference.

### BACKGROUND OF THE INVENTION

This invention relates to the general field of endoscopic medical devices and specifically to those devices used for ablation of lesions and control of bleeding using bipolar or monopolar cautery techniques in the medical field.

The use of heat for the cauterization of tissue dates to ancient times. In the present century the use of radio frequency (RF) electrical current traveling through a portion of the body has been widely used to stop bleeding. Cauterization of tissue arises by virtue of its resistance to the passage of RF electrical energy. In the cauterization of bleeding, the proteins in the tissue are heated to a temperature where the proteins congeal and the walls of bleeding vessels are welded together to stop the bleeding. RF electrical energy is preferred because its frequency is above that which could otherwise cause neuro-muscular stimulation. Several modes of RF cauterization of tissue are employed, such as monopolar or bipolar coagulation.

In monopolar coagulation, an active electrode of small dimensions such as of the order of one to two mm is applied to the bleeding site and the current path is completed through the body to a distal end electrically in contact with a large surface area of the body such as the buttocks or back. One technique in which the monopolar mode may be employed involves fulguration which is the use of a spark or arc from the active electrode to the tissue. In bipolar coagulation, the two active electrodes are closely spaced, of the order of millimeters so that the current path is confined to a local region of the tissue.

In the medical field, to provide care to patients, there is often a need to ablate lesions that may include dilated blood vessels (vascular malformations), neoplastic lesions (early cancers) or control bleeding from blood vessels that have been eroded and exposed by invading stomach or duodenal ulcers. These lesions are usually located deep within the body and cannot be easily reached except with specialized instruments such as endoscopes. Specialized versions of endoscopes for use in particular areas of the body or particular procedures include but are not limited to colonoscopes, bronchocopes, cystoscopes, laparoscopes, and sigmoidoscopes.

Typically these specialized endoscopes are of thin caliber because they need to be passed via small natural orifices (mouth, rectum, nares, urethra) along the thin internal passageways to the point of interest where the lesion is located. For example, the endoscope that is used to evaluate the upper gastrointestinal tract (UGI tract) measures 9 mm in diameter and is 140 cm in length and can be passed via the mouth to evaluate the UGI tract including the esophagus, stomach and duodenum. Similarly the colonoscope which is used to evaluate the colon measures 12-13 mm in diameter and is 180 cm in length and it can be passed through the rectum and used to evaluate the entire colon and terminal ileum. These specialized endoscopes typically all have a small internal channel that runs the length of the endoscope to allow the manipulating physician to pass instruments from the exterior through the entire length of the endoscope all the way to the tip of the endoscope and a little beyond the end of the scope to obtain biopsies, resect lesions, ablate lesions and cauterize lesions that are located deep within the body.

The working channel of these specialized endoscopes are of very small caliber and can usually only accommodate accessories that have a diameter of 3.2 mm or less. Quite often during endoscopy and colonoscopy lesions are encountered that need to be ablated by electrocautery technique. The ablation of these lesions usually requires the use of a monopolar or bipolar cautery probe that is passed via the working channel of an endoscope into the internal part of the body of the patient to the sight of the lesion. Typically these probes are long (180 cm or more) and of thin caliber 2.2-3.2 mm. All the cautery probes available for use in an endoscope are limited in size to 3.2 mm or less because this is the maximum diameter of the working channel of the endoscope. Quite often however the lesions encountered are large blood vessels measuring 5 mm or more in size and require a cautery probe of larger diameter to effectively, easily and safely ablate the lesion. Similarly bleeding vessels seen in the base of eroding gastric or duodenal ulcers are of diameter 4-5 mm and can be very difficult to ablate using the standard 3.2 mm cautery probe due to the size discrepancy between the instrument and the lesion. The limitation in the size of the tip of the cautery probe also increase the time it takes to ablate the lesion and also increases the likelihood of incomplete ablation and subsequent complications. In addition due to the limitation in the size of the cautery probes one other disadvantages of the cautery probes is the cylindrical cross-section and flat tip that limits the ability to achieve close apposition to the tissue to be ablated. Since the interior of the GI tract has a concave configuration when viewed inside, tangential application of the cylindrical bipolar cautery probe often does not provide effective tissue contact and hemostasis.

Although, existing cautery devices are useful, they often do not provide satisfactory operation for a number of reasons as outlined above.

### SUMMARY OF THE INVENTION

The present invention is for an expandable cautery device. The device uses a probe having a shaft having a cauterizing end thereon. The cauterizing end is expandable. The cauterizing end may define a chamber that forms a balloon at the distal end thereof. Fluid is introduced into the chamber to expand the cauterizing end. The balloon has either a single electrical contact in a monopolar configuration or a plurality of different polarity electrical contacts on the surface of the balloon in a multipolar configuration. The contacts are connected to a power source. In the monopolar configuration a grounding pad is used to complete an electrical circuit, and when the probe of the cautery device contacts tissue to be treated electrical resistance will provide a cautery effect. In the multipolar configuration when contacts of opposite polarity contact tissue to be treated a short circuit is created between them, and electrical resistance within the tissue provides a cautery effect. The probe is introducible through the bore of an endoscope, but the probe itself may be introduced without the use of an endoscope.

### BRIEF DESCRIPTION OF THE DRAWINGS

FIG. 1 is an end view of the probe;

FIG. 2 is a two dimensional view of the contacts before the contacts are applied to the balloon;

FIG. 3 is a section view showing the cautery device of this invention inside the stomach;

FIG. 4 is a perspective view of the multipolar probe in a deflated state;

FIG. 5 is a perspective view of the probe extending from the distal end of an endoscope, the probe is in an inflated state;

FIG. 6 is a perspective section view of the probe in FIG. 5;

FIG. 7 is a perspective view of the monopolar probe in a deflated state; and

FIG. 8 is a perspective view of the monopolar probe shown in FIG. 7 in an inflated state projecting from the distal end of an endoscope.

### DETAILED DESCRIPTION OF INVENTION

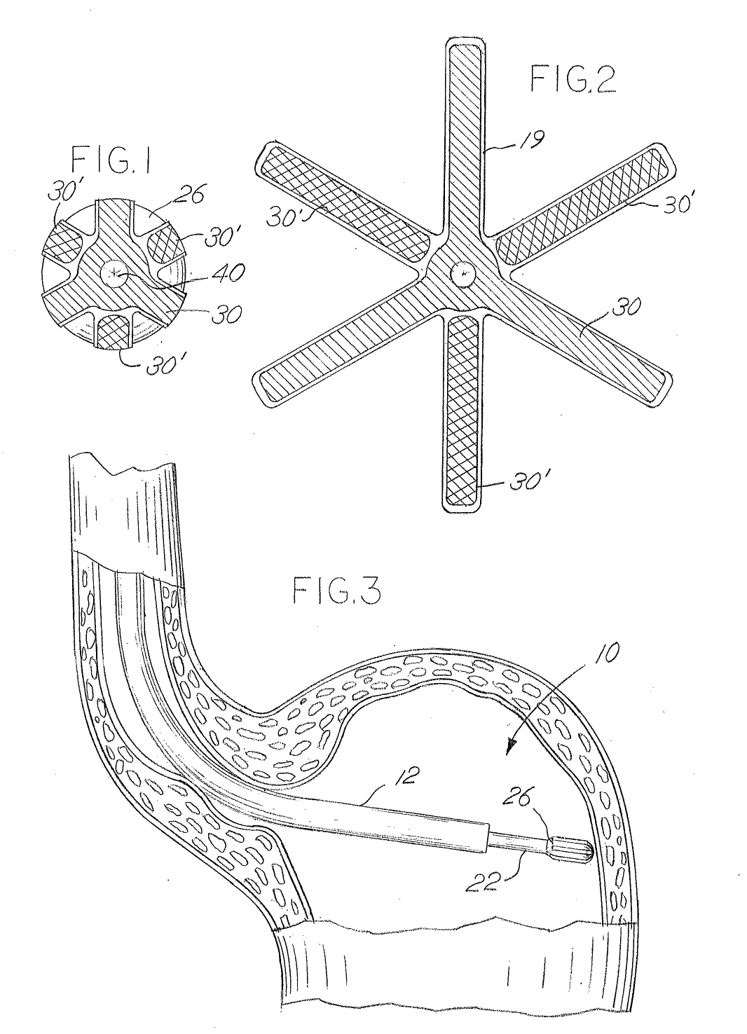

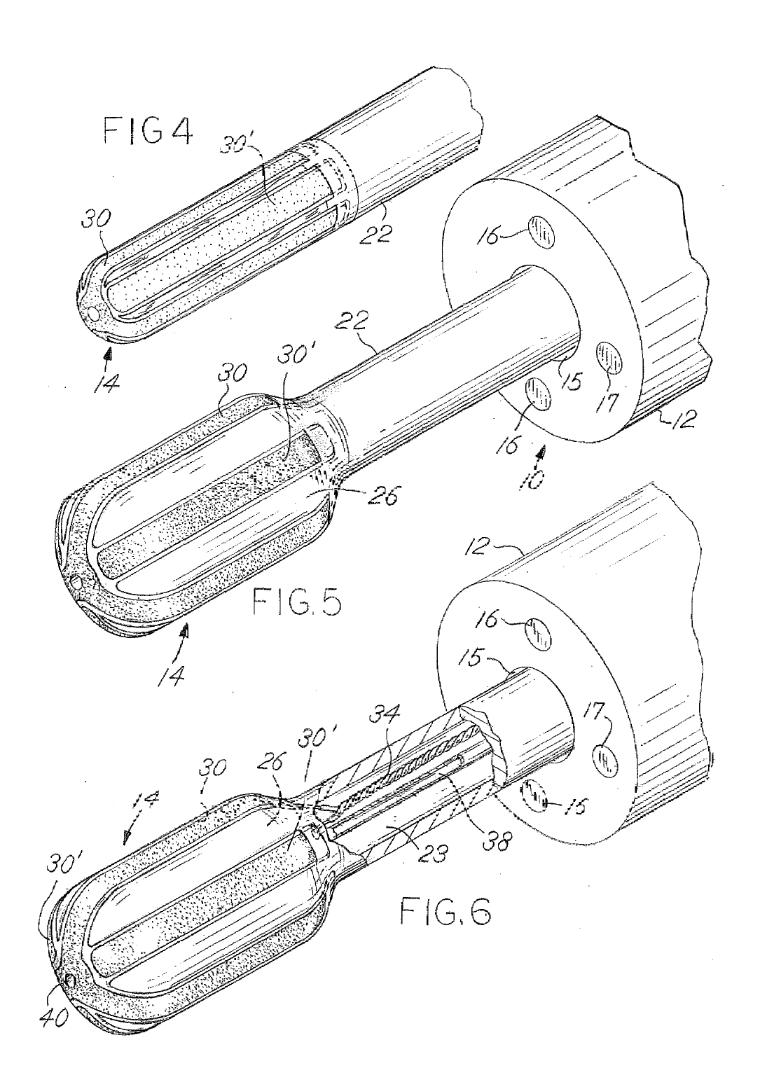

The expandable cautery device 10 of this invention has a tube which is typically an endoscope 12, and a probe 14. The endoscope has a bore 15 and contains a pair of light sources 16 and a sensor 17 for delivering an image to a physician using the endoscope 12. The probe 14, which is shown in FIGS. 1 and 4, has a flexible shaft 22 which is attached to an expandable balloon 26. The shaft 22 has a first lumen 23 extending through the entire length of the shaft 22 and connected to the balloon 26. The balloon 26 is an inflatable chamber at the distal end of the shaft 22 and connected to the first lumen 23. The shaft 22 and balloon 26 are slidable within the bore 15 of the endoscope and may be retracted into the bore 15 of the endoscope 12. The balloon 26 forms a cauterizing end that includes electrical contacts 30.

In a first embodiment electrical contacts 30 may be arranged as shown in FIG. 2. Electrical contacts 30 of opposite polarities are separated from each other by an appropriate distance to prevent arcing between the contacts 30. The different polarities are illustrated by the different hatching shown in FIG. 2. The contacts 30 may be made of a conductive metal, conductive polymer, or a painted conductor on the surface of the balloon 26. In the configuration of contacts 30 as shown in FIG. 3 the contacts are attached to a thin polymer sheet 19. The polymer sheet 19 may be made of polyimide. Contacts of one polarity are joined together as a continuous, thin piece of conductive metal. Contacts 30 of the opposite polarity are shown as discontinuous pieces of metal. The discontinuous contacts 30′ connected together by jumper wires or a ring near where the balloon 26 meets the shaft 22. When metal contacts 30 are used, the ends of the contacts 30 nearest the shaft 22 are embedded near where the balloon 26 joins the shaft 22 to prevent the contacts 30 from peeling from the surface of the balloon 26. Wires 34 are connected to the electrical contacts 30 and extend through the first lumen 23 to a remote power source. FIG. 6 shows the wires 34 in the first lumen 23. The wires 34 are insulated along their entire length until they are connected to contacts 30 on the balloon 26. Contacts 30 of different polarity are selectively sized and generally uniformly distributed in spaced apart pairs of opposite polarity, over the balloon 26. FIGS. 1 and 2 show a common arrangement for the contacts 30. The contacts 30 may also be spirally arranged, or transversely arranged over the entire surface of the balloon 26. The ratio of the width of the contacts 30 to the spacing between them is selected so as to provide, a predetermined minimum number of spaced apart pairs of electrodes and to allow omnidirectional multipolar treatment of tissue when the probe 14 is projected from the distal end of the endoscope 12. The term multipolar, as used herein, means the electrosurgical use of a plurality of contacts 30 which are arranged in fixed relationship with each other on a probe 14 for at least a bipolar contact with a precise treatment of tissue targets over a wide range of orientations of the device relative to the tissue target.

The balloon 26 can be made of an elastic material or a non-elastic material. The material should have heat tolerance to at least 100 degrees centigrade as this is the temperature required for tissue cautery effect. If the balloon is elastic, it will conform to a lesion and distribute pressure evenly to the zone to be coagulated without leaving gaps between the balloon 26 and the lesion. If the balloon 26 is a foldable non-elastomeric balloon it will be rigid when inflated and provide for excellent tamponade of the tissues which is very helpful when trying to control bleeding from leaking blood vessels in the gastrointestinal tract. The elastic balloon may be made of silicon rubber, which is flexible, does not stick, and can tolerate high temperatures of 100 degrees centigrade or more. A non-elastic balloon may be made of engineering plastic that can tolerate high temperatures such as polytetrafluroethylene (PTFE) or perfluoroalkoxy fluorocarbon (PFA) or fluoroethylene-propylene (FEP) or polyethylene terephthalate (PET) etc. These engineering plastics can tolerate high temperatures and are flexible but non-elastomeric. The exterior of the balloon 26 may be coated with a non-stick coating having a low coefficient of friction, such as silicone, teflon or polysiloxane.

The first lumen 23, containing wires 34, is used to fill the balloon 26 with fluid supplied from a source remote from the distal end of the shaft 22. The remote source connected to the first lumen 23 is usually a standard syringe filled with a fluid preferably saline or water. When the probe 14 is used with an endoscope 12 it is typically fed through the bore 15 of the endoscope 12 until it emerges from the distal end of the endoscope 12. When the balloon 26 is not inflated it has a diameter similar to that of the bore 15 of the endoscope 12. Endoscope 12 diameters are typically between 2.8 and 3.2 mm, although in some cases larger diameters may be available. When the balloon 26 is beyond the distal end of the endoscope 12 the fluid is injected into the first lumen 23, the fluid is communicated through the first lumen 23 into the balloon 26 causing inflation of the balloon 26. Inflation of the balloon 26 can make the balloon 26 much larger than the bore 15 of the endoscope 12. When the balloon 26 makes contact with tissue to be treated a short circuit is created between contacts 30 of opposite polarities thereby producing an electrocautery effect. This allows a user of the probe 14 to confine heat to the tissue to be treated since heat is not generated in areas of the probe 14 not in contact with tissue.

A second lumen 38 may be included in the probe 14. The second lumen 38 runs the entire length of the shaft 22 and is shown in FIG. 6. The second lumen 38 terminates in an outlet 40 at the end of the balloon 26. Water may be introduced into the second lumen 38 from an external source so that the water is communicated through the second lumen 38 and discharged at the outlet 40. An alternative use of the second lumen 38 is that the probe 14 may be placed on a guide wire extending through the length of the second lumen 38, which is known to those skilled in the art as a way to position a probe without the use of an endoscope 12.

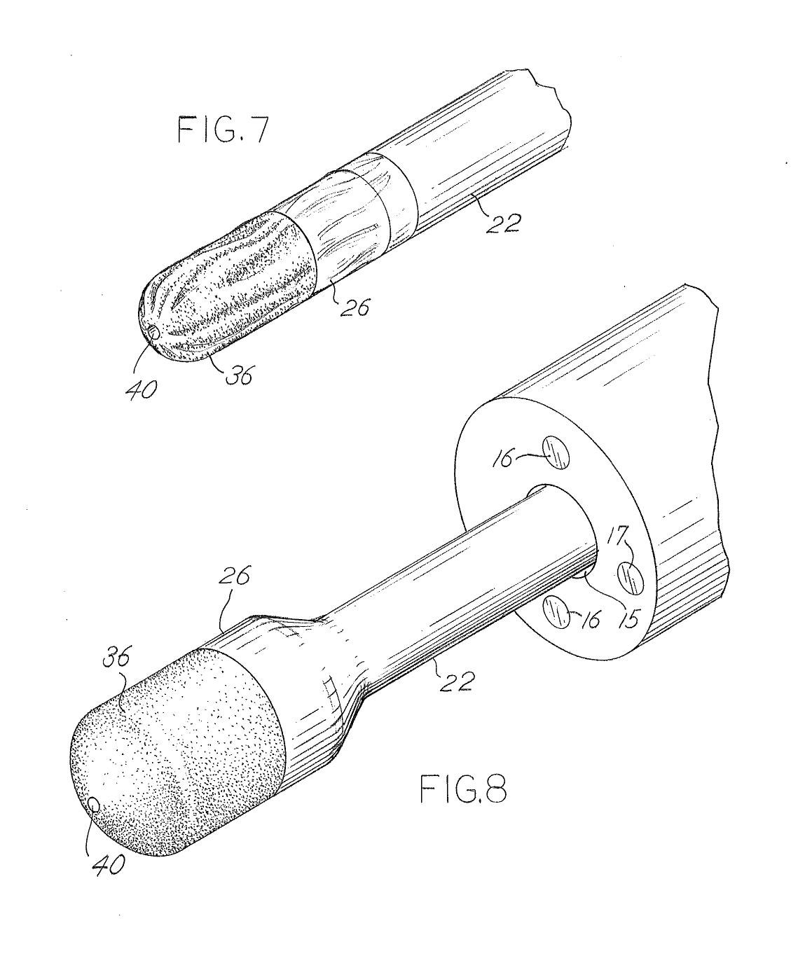

A second embodiment of the invention is a monopolar cautery probe 14 and has a single contact 36 on the balloon 26 as shown in FIGS. 7 and 8. A grounding pad located on the body of a patient provides a second contact of opposite polarity to the contact 36 on the balloon 26. A single wire extends through the length of the shaft 22 and terminates in a monopolar electrode that covers a substantial portion of the balloon 26. The electrical circuit is completed through the much larger contact area of the grounding pad. The small contact area of the balloon 26 and the tissue to be treated compared to the large surface area between the grounding pad result in high resistance and a thermal electrocautery effect where the balloon 26 makes contact with the tissue. The monopolar embodiment is similar to the first multipolar embodiment in function and structure aside from the fact that the cautery effect is provided through only one electrical contact 36 covering most of the surface of the balloon 26.

In both embodiments the probe 14 is used for engagement with and treatment of body tissue on the basis of tissue conduction of RF electrical energy and subsequent thermal effect. The probe 14 is sized and constructed for insertion into the body of a patient through the bore 15 of an endoscope as shown in FIG. 3. Specialized endoscopes 12 may consist of colonoscopes, bronchocopes, cystoscopes, laparoscopes, and sigmoidoscopes, but are not limited to such devices. Although the invention is introducible through an endoscope 12, it is possible to use a smaller tube that does not contain a scope. In certain applications the probe 14 may be introduced into a patient alone without the use of any tube. The probe 14 is designed to pass through the bore 15 of an endoscope. When the balloon 26 is extended beyond the end of the endoscope 12 as shown in FIGS. 3, 5, 6, and 8 the balloon 26 it may be filled with fluid entering through the first lumen 23 and supplied by the remote source such as a standard syringe. The fluid is preferably saline or water, but may be air. Saline and water are incompressible and provide a user with greater ability to apply tamponade pressure when using the probe 14. The balloon 26 has an inflated shape that is substantially spherical and larger than the bore 15 of the endoscope 12.

The probe is designed to be used with standard and readily available electrical generators. A standard electrosurgical generator such as ERBE or Valley Forge may be connected to two, pin leads attached to the wires 34.

The probe 14 is insertable through a natural orifice into a patient's body as shown in FIG. 3 using an endoscope, or through the use of a guidewire running the length of the second lumen 38. As shown in FIG. 3, once the endoscope 12 has been inserted into the patient's body, it is used for viewing the inside of internal organs such as the stomach, other parts of the gastrointestinal system, lung, etc., to determine the location of a bleeding lesion. If necessary water may be introduced through the second lumen 38 which will project water from the outlet 40 and cleanse the area to be treated. The probe 14 in a deflated state is inserted through the bore 15 of an endoscope 12 and is extended beyond the distal end of the endoscope 12. The balloon 26 is inflated with saline, water, air. The user then selects the wattage on the generator. For example a typical setting of 15-30 watts is used to treat a stomach ulcer. The balloon 26 is placed against the lesion and RF electrical current is passed through the probe 14 to provide that results in restive conduction through tissue to be treated. This in turn leads to the generation of electrical potential through local tissues in contact with the monopolar contact 36 or bipolar contacts 30 that results in a coagulative ablative effect. The combination of mechanical pressure and thermal coagulation causes a welding together of the walls of bleeding vessels and can control bleeding. The combination of heat and pressure causes coagulaton of lesions treated. The tissue coagulation zone is not limited to the size of the bore 15 of an endoscope 12. Because the balloon 26 expands to larger than the bore 15 of the endoscope 12 a larger area may be treated with only one application. This eliminates the need to touch and retouch many times with smaller probes. Also due to the substantially spherical shape of the inflated balloon 26 it is possible to approach tissue to be treated from any angle. Therefore, it is possible to treat tissue end-on or obliquely.

With a multipolar device in accordance with this invention, the electric field pattern around the balloon 26 may be selected to provide a desired shape and depth. In some applications where a lesser radial electrical field and depth of injury is desired to reduce the depth of coagulation, the gap between the contacts 30 may be reduced. In such case a larger number of closely spaced contacts 30 can be employed. When a deeper tissue treatment is needed, the gap or space between contacts 30 may be increased. The width of contacts 30 and gap sizes may thus be selected, depending upon the particular tissue being treated.

Some of the considerations in the selection of the width of contact 30 (W) to spacing of contact 30 (S) ratio relate to the heat distribution achieved in the tissue to be treated and the generation of tissue sticking problems. For example, a tissue sticking problem arises when a high concentration of heat causes too high a temperature in the tissue, generally greater than about 200 degree Fahrenheit, thus resulting in the adherence of tissue to metal parts of the probe 14. If such condition occurs, the probe body requires frequent removal for cleaning and undesirably extends the duration of the treatment of the patient. When such excessive amount of heat is applied to stop a bleeding area, the resulting sticking of cauterized tissue also makes it difficult to disengage the probe body without removing the coagulated layer and restarting bleeding.

Preferably, just enough electrical power, generally in the range from about 10 watts to about 30 watts for a 2.3 mm diameter probe, should be applied to thermally coagulate the tissue area in contact with the probe to stop bleeding. The electrical power further should be applied in such manner that high voltage punch-through of cauterized dried tissue leading to sticking and/or unnecessary tissue wall damage is avoided. The electrical power normally is supplied in pulses having a duration of the order of one or several seconds.

Tissue sticking problems can be substantially avoided with a multipolar device in accordance with this invention because it enables the application of an adequate amount of electrical power at a relatively low voltage. The amount of power that can be applied is a function of the surface area of the probe 14 and contacts 30 brought into contact with the tissue. When the surface area is relatively large, i.e. with an adequate conductor or electrode width, W, to spacing, S, ratio, there exists sufficient surface contact between an electrode and the tissue to supply electrical power at a relatively safe low voltage which is unlikely to force power through a dessicated layer causing deeper damage and risk of perforation.

The contact 30 to tissue contact area tends to be a function of the ratio of the contact width, W, to the spacing, S between contacts. At a low ratio, say less than about 1:3 or expressed in a fraction ⅓, the minimum amount of power needed to stop bleeding requires a voltage that is likely to be above the safe operating range. At such lower W:S ratio of about ⅓ the multipolar probe may provide the desired coagulating function; however, the impedance or resistance between the probe and tissue with such low ratio tends to be higher because the conductor surface in contact with tissue is less, thus requiring a higher voltage to transfer the desired amount of power into the tissue. This higher voltage tends to result in less uniform heating with hot spots that are likely to cause tissue sticking.

The W:S ratio, of the contact 30 width, W, to spacing, S, thus should be greater than about one-third (⅓) below which value less uniform heating with likelihood of sticking tends to occur. Preferably the W:S ratio is not less than about one-half (½). At W:S ratios of about 1:1 and 2:1 the probe tends to function adequately with good uniform heating. With a W:S ratio of 3:1, or expressed as 3, there is a tendency for less uniform heating but the presence of a relatively larger contact 30 surface area enables operation at a lower voltage which is safer from a standpoint of avoiding tissue sticking. Generally W:S ratios ranging from 1:1 to 1:2 is preferred.

Variations from the described embodiments may be made by one skilled in the art without departing from the scope of the invention. The above described invention is not to be limited to the details given but may be modified within the scope of the following claims.

What is claimed is:

1. An expandable cautery device comprising:
a tube having a distal end, said tube having a bore along its entire axis;
a probe having a shaft with a cauterizing end thereon, said shaft and cauterizing end slidably received within said bore, said cauterizing end extendable from said distal end of said tube, and said cauterizing end being expandable.

2. An expandable cautery device as claimed in claim 1, wherein said shaft defines a first lumen through the entire length of said shaft, said cauterizing end defines a chamber in communication with said first lumen, said first lumen receiving fluid from an external source and communicating said fluid to said cauterizing end to inflate said cauterized end.

3. An expandable cautery device as claimed in claim 1, wherein said cauterizing end includes electrical contacts on an exterior surface thereof, said electrical contacts having different polarities, said electrical contacts connected to a power source.

4. An expandable cautery device as claimed in claim 1, wherein said tube is an endoscope, said endoscope including a light source near said distal end and a light sensor for providing an image of an area near said distal end of said endoscope.

5. An expandable cautery device as claimed in claim 1, wherein said cauterizing end includes an electrical contact having one polarity at said cauterizing end and another contact of another polarity adapted for grounding on a patient.

6. An expandable cautery device as claimed in claim 2, wherein said shaft contains a second lumen through the entire length of said shaft, said second lumen terminating in an outlet on said cauterizing end, said second lumen transporting fluid through said shaft to be expelled from said outlet.

7. An expandable cautery device comprising:
a tube having a distal end, said tube having a bore along its entire axis;
a probe having a shaft with a cauterizing end thereon, said shaft and cauterizing end slidably received within said bore, said cauterizing end extendable from said distal end of said tube, said cauterizing end terminating a balloon, said balloon being expandable when exposed from the distal end of said tube.

8. An expandable cautery device as claimed in claim 7, wherein said tube is an endoscope, said endoscope including a light source near said distal end and a light sensor for providing an image of an area near said distal end of said endoscope.

9. An expandable cautery device as claimed in claim 7, wherein said balloon in made of a non-elastic material, said balloon expandable to a predetermined size when filled with fluid, and said balloon collapsible when said fluid is withdrawn.

10. An expandable cautery device as claimed in claim 7, wherein said balloon is made of an elastic material, said material stretchable such that balloon expands from an initial size when fluid is introduced into said balloon and said balloon returns to said initial size when said fluid is withdrawn from said balloon.

11. An expandable cautery device as claimed in claim 7, wherein said balloon includes electrical contacts on an exterior surface thereof, said electrical contacts having different polarities, said electrical contacts connected to a power source.

12. An expandable cautery device as claimed in claim 7, wherein said balloon includes an electrical contact having one polarity on an exterior surface of said balloon and another contact of another polarity adapted for grounding on a patient.

13. An expandable cautery device as claimed in claim 7, wherein said shaft contains a second lumen through the entire length of said shaft, said second lumen terminating in an outlet on said cauterizing end, said second lumen transporting fluid through said shaft to be expelled from said outlet.

14. An expandable cautery device comprising:
a probe having a shaft with a cauterizing end thereon, said cauterizing end being expandable.

15. An expandable cautery device as claimed in claim 14, wherein shaft defines a first lumen through the entire length of said shaft, said cauterizing end terminating in a balloon which defines a chamber in communication with said first lumen, said first lumen receiving fluid from an external source and communicating said fluid to said balloon to inflate said balloon.

16. An expandable cautery device as claimed in claim 15, wherein said balloon includes at least one electrical contact on an exterior surface thereof, said electrical contact connected to a power source.

17. An expandable cautery device as claimed in claim 16, wherein said electrical contact on said balloon is one polarity and another contact of another polarity is adapted for grounding on a patient.

18. An expandable cautery device as claimed in claim 16, wherein said balloon contains a plurality of electrical contacts on said exterior surface of said balloon, some of said contacts having different polarities.

19. An expandable cautery device as claimed in claim 18, wherein said contacts on said surface of said balloon are positioned such that when said probe contacts tissue at any angle to said tissue a short circuit will occur between said contacts of different polarities thereby cauterizing said tissue.

20. An expandable cautery device as claimed in claim 16, wherein said shaft contains a second lumen through the entire length of said shaft, said second lumen terminating in an outlet on said cauterizing end, said second lumen transporting fluid through said shaft to be expelled from said outlet.

21. An expandable cautery device as claimed in claim 16, wherein said balloon is made of a non-elastic material.

22. An expandable cautery device as claimed in claim 16, wherein said balloon is made of an elastic material, said material stretchable such that balloon expands from an initial size when fluid is introduced into said balloon and said balloon returns to said initial size when said fluid is withdrawn from said balloon.

23. An expandable cautery device as claimed in claim 16, wherein said shaft contains a second lumen through the entire length of said shaft, said second lumen terminating in an outlet on said cauterizing end, said second lumen adapted for slidably receiving a guidewire for positioning said probe.
